(19) United States
(12) Patent Application Publication
(10) Pub. No.: US 2007/0231373 A1
(43) Pub. Date: Oct. 4, 2007

(54) Transdermal Steriod for Formulation

(75) Inventors: James Robert Murray, Bristol (GB); Fumio Kamiyama, Kyoto (JP); Ernst Wulfert, Brussels (BE)

Correspondence Address: GREENLEE WINNER AND SULLIVAN P C, 4875 PEARL EAST CIRCLE, SUITE 200, BOULDER, CO, 80301, US

(73) Assignee: HUNTER-FLEMING LIMITED, Bristol (UK)

(21) Appl. No.: 11/568,387

(22) PCT Filed: Apr. 28, 2004
(86) PCT No.: PCT/GB05/01596

§ 371 Date: Mar. 13, 2007

(30) Foreign Application Priority Data
Apr. 28, 2004 (GB) 0409498.3

(51) Int. Cl. A61K 31/57; A61K 31/56; A61K 9/70

(52) U.S. Cl. 424/449; 514/169; 514/177

(57) ABSTRACT

Compounds of formula (I) (in which R¹; R², R³ and R⁴ are the same as or different from each other and each represents an oxo group, a hydroxy group, a mercapto group, a hydrogen atom, a halogen atom, an alkoxy group or an aryloxy group; and the dotted line indicates that there may be a single or double bond between one of the respective pairs of carbon atoms) and esters thereof can be administered by transdermal administration. They are particularly effective when applied as a patch, preferably with an adhesive comprising a copolymer of from 40 to 60% by weight of methoxyethyl acrylate, from 30 to 40% by weight of lauryl acrylate or lauryl methacrylate and from 10 to 25% by weight of a polar monomer.

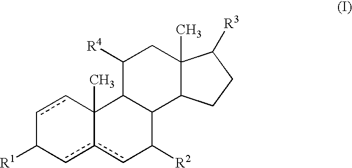

The present invention relates to a transdermal formulation for the treatment, inter alia, of osteoporosis and related ailments.

Osteoporosis, a condition in which bone mass decreases, resulting in the bone losing some of its structural integrity, is a condition particularly prevalent in post-menopausal women. It is often treated by the administration of steroids, such as oestrogen.

EP698612 discloses that certain 11-hydroxy steroids are particularly useful for the treatment and prevention of osteoporosis and for osteogenesis, without many of the side effects of known treatments. There is no indication in this specification as to the route by which the steroids are to be administered. However, the Examples show the use of the compounds by injection.

WO 02/00224 and WO 02/00225 disclose the use of certain 7-hydroxy-steroid compounds, especially 3-hydroxy-7β-hydroxy-steroid compounds, for protection against neuronal cell death. These suggest that, in order to achieve a rapid effect, the compounds should be administered by intravenous injection.

WO 03/015791 discloses the use of 3-hydroxy-7-hydroxy steroids and 3-oxo-7-hydroxy steroids, especially the 7β-isomers thereof, and pharmaceutically acceptable esters thereof for protection against ischaemia-induced damage to peripheral organs, such as the heart or kidneys.

Although the two most common methods of administering medication are orally or by injection, both methods have serious disadvantages. Oral administration of a drug depends on the drug being stable to the acid environment of the stomach as well as to the intestinal mechanism, or on the drug being protected from that environment. Moreover, people differ in the rate and manner in which drugs are absorbed, and so the amount of the drug actually reaching the bloodstream via the oral route can vary enormously from person to person.

Injection avoids these disadvantages but has to be done by a skilled or trained person, is often disliked, sometimes intensely, by the patient and can cause damage if done too often.

Traditionally, transdermal administration (i.e. administration through the skin, but without breaking the skin) has been used only for those drugs which affect the skin or immediately underlying muscle tissue. However, it can be used with other drugs provided that those drugs can pass through the skin sufficiently efficiently and sufficiently predictably.

We have now surprisingly found that transdermal delivery can be used with certain steroids, including those of EP698612, WO 02/00224 and WO 02/00225.

Thus, the present invention consists in the use of compounds of formula (I): 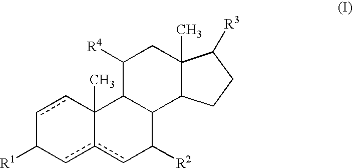
(in which R¹; R², R³ and R⁴ are the same as or different from each other and each represents an oxo group, a hydroxy group, a mercapto group, a hydrogen atom, a halogen atom, an alkoxy group or an aryloxy group; and the dotted line indicates that there may be a single or double bond between one or two of the respective non-adjacent pairs of carbon atoms) and esters thereof for the manufacture of a medicament for transdermal administration.

The invention also consists in a pharmaceutical formulation for transdermal administration having as the active ingredient a compound of formula (I), as defined above, or an ester thereof.

The invention further consists in an adhesive patch for the transdermal administration of a compound of formula (I) or an ester thereof, as defined above, said patch comprising a substrate and a layer of adhesive on the substrate, said adhesive having dispersed therein at least one compound of formula (I) or an ester thereof.

Among the compounds of the present invention, preferred compounds include those compounds of formula (II): 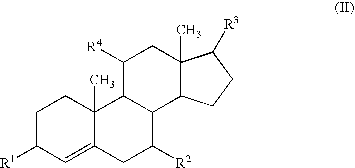
(in which R¹, R², R³ and R⁴ are as defined above) and esters thereof.

Another preferred class of compounds of the present invention are those compounds of formula (III): 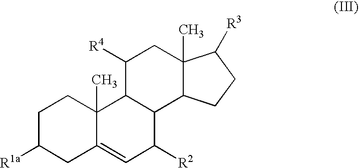
(in which R^{1a} represents an oxo group, a hydroxy group, a mercapto group or a halogen atom; and R², R³ and R⁴ are as defined above) and esters thereof.

A still further preferred class of compounds of the present invention are those compounds of formula (IV): 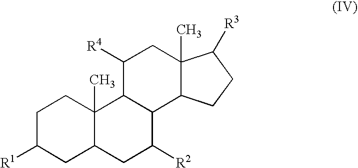
(in which R¹, R², R³ and R⁴ are as defined above) and esters thereof.

Examples of compounds of formula (II) include 11β-hydroxy-4-androstene-3,17-dione, which has the formula (IIa): 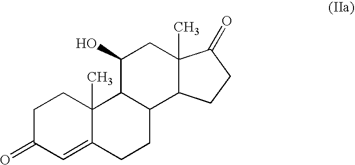
and esters thereof. This compound, which is the preferred compound for use in the present invention, is hereinafter referred to as “HAD”.

Examples of compounds of formula (III) include 7α-hydroxy-dehydroepiandrosterone (7α-hydroxy-DHEA), which has the formula (IIIa): 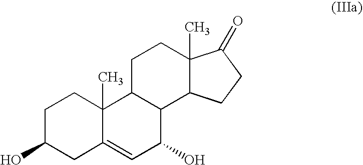
and esters thereof.

Examples of compounds of formula (IV) include 7β-hydroxy-epiandrosterone, hereinafter referred to as 7β-OH EPIA, which has the formula (IVa): 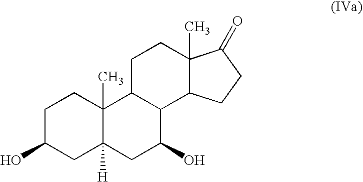
and esters thereof.

In the compounds of the present invention, where R¹, R^{1a, R}², R³ or R⁴ represents a halogen atom, this may be a fluorine, chlorine, bromine or iodine atom, preferably a chlorine atom.

Other useful steroids which may be used as the compound of formula (I) include such androgens as testosterone and 3β,17β-dihydroxyandrostane.

Of the compounds referred to above, the most preferred are 7α-hydroxy-dehydroepiandrosterone, 7β-hydroxy-epiandrosterone (7β-OH EPIA) and 11β-hydroxy-4-androstene-3,17-dione (HAD).

Patches for transdermal administration of medication normally comprise a substrate of a medically acceptable fabric or film on which is a layer of an adhesive impregnated with the medication. Any substrate commonly used in the field may be used in the present invention. It may, for example, be a woven fabric, non-woven fabric, porous film or moulded film, ideally 10-100 μm thick. Woven fabrics, non-woven fabrics and porous films are useful in that they allow the passage of water vapour, while moulded films are useful for providing a barrier to bacteria and for water-proofing.

However, it will be appreciated that the nature of the adhesive may have a significant effect on the utility of the patch, and so this needs to be chosen with some care. Thus, the adhesive must be capable of adhering to the skin, it must permit sustained drug release and it must not cause irritation to the skin. In general, it must maintain these properties over a relatively long period, e.g. 3 to 7 days, after attachment, despite the normal flexing of the skin and possibly washing. Moreover, the adhesive must not be so strong that it causes damage to the skin when the patch is removed.

We have now found that a particular class of adhesives is especially useful with the steroid compounds of formula (I) and esters thereof used in the present invention.

These new adhesives comprise a copolymer of from 40 to 60% by weight of methoxyethyl acrylate, from 30 to 40% by weight of lauryl acrylate or lauryl methacrylate and from 10 to 25% by weight of a polar monomer.

The amount of units derived from methoxyethyl acrylate in the copolymer should be from 40 to 60% by weight, more preferably from 45 to 55% by weight. If this amount is greater than 60% by weight, gelling occurs during polymerisation and the resulting copolymer becomes insoluble. Moreover, the viscosity of the copolymer is poor. On the other hand, if the amount of methoxyethyl methacrylate is less than 40%, drug solubility in the adhesive is reduced and the relatively high drug loadings otherwise achievable are not achieved.

The second co-monomer, lauryl acrylate and/or lauryl methacrylate, is present in an amount of from 30 to 40% by weight of the monomer units in the copolymer. Its presence enhances the adhesion of the adhesive and, when combined with the methoxyethyl acrylate, it provides suitable conditions for dissolving the steroids used in the present invention as well as absorption promoters, where these are used. If the amount of lauryl acrylate and/or lauryl methacrylate units in the copolymer is less than 30% by weight, adequate hydrophobicity is not achieved. On the other hand, if the amount is greater than 40%, the copolymer becomes too hydrophobic and drug solubility is reduced.

If 2-ethylhexyl acrylate or 2-ethylhexyl methacrylate were used in place of the lauryl analogue, since the chain length of the 2-ethylhexyl group is less than that of the lauryl group, the ability of the resulting copolymer to dissolve hydrophobic drugs and absorption promoters would be reduced. On the other hand, if stearyl acrylate or methacrylate, with a longer chain length, were used in place of the lauryl analogue, the adhesion of the adhesive would be poor.

The third necessary monomer in the copolymers used in the present invention is a polar monomer. This serves to impart to the copolymer higher cohesion without damaging the other desirable properties mentioned above and it can improve the cross-linking of the polymer. It should be present in the copolymer in an amount of from 10 to 25% by weight of the copolymer units. Less than 10% by weight of the polar monomer will not give adequate cohesion, while more than 25% will result in the copolymer becoming too polar and the adhesiveness being reduced. Examples of suitable polar monomers include acrylic acid, methacrylic acid, acrylamide, methacrylamide, N-vinyl-2-pyrrolidone, N,N-dimethylacrylamide, 2-hydroxyethyl acrylate and vinyl acetate. A single one of these or a combination of two or more may be used. Of these, acrylic acid, N-vinyl-2-pyrrolidone and 2-hydroxyethyl acrylate are preferred, N-vinyl-2-pyrrolidone being most preferred, either alone or in combination with at least one other polar monomer. In any event, we particularly prefer that units derived from N-vinyl-2-pyrrolidone should constitute at 5% by weight of the copolymer, provided that the total amount of polar monomer is, as stated above, from 10 to 25% by weight of the copolymer.

As mentioned above, an absorption promoter may be included in the adhesive composition according to the present invention. Examples of such absorption promoters include: fatty acid esters, such as isopropyl palmitate and isopropyl myristate; glycerol esters, such as glyceryl monolaurate and glyceryl monooleate; acid amides, such as lauric acid diethanolamide; and neutral surfactants, such as polyethylene glycol dilauryl ether. However, such materials are well known in the art, and any common absorption promoter may be used. The preferred absorption promoter is isopropyl myristate. Clearly, the amount of the absorption promoter must be sufficient to enhance transdermal absorption of the drug. However, too much may have the effect of reducing the adhesiveness of the patch. Accordingly, we prefer that the amount of absorption promoter should be from 3 to 40 parts by weight per 100 parts by weight of the copolymer.

It is also preferred that the copolymer used in the adhesive composition should be cross-linked by means of a cross-linking agent in order to enhance cohesion. Examples of such cross-linking agents include isocyanates and chelating agents. The amount of the cross-linking agent used is preferably from 0.1 to 2 parts by weight. If the amount used is less than 0.1 parts by weight, little cross-linking takes place and there is no significant benefit from its addition. On the other hand, if the amount added is more than 2 parts by weight, adhesiveness is reduced.

The copolymer may be prepared by methods well known in the art for the preparation of polymers of this type, for example by free radical polymerisation using the monomers defined above. Any conventional process, such as solution polymerisation, suspension polymerisation or emulsion polymerisation, may be used. Solution polymerisation is particularly preferred, since the resulting molecular weight distribution is relatively narrow and there is, as a result, little variability in adhesiveness.

Surprisingly, we have found that the copolymers suggested above, when used as the adhesive, actually enhance the absorption of the compounds of formula (I) through the skin.

The active ingredient may be mixed with the adhesive copolymer by adding it, and, if desired, an absorption promoter and/or a cross-linking agent, to a solution containing the copolymer in a suitable solvent. The resulting mixture may then be laminated onto the desired support using, for example, a knife or roll coater, and then dried in an oven, for example at a temperature of from 50 to 100° C., for a suitable period to remove the solvent and cause the adhesive composition containing the active ingredient to adhere to the substrate.

The amount of the mixture applied to the substrate is preferably such that the thickness of the composition after drying is from 30 to 120 μm. If the thickness is less than 30 μm, adhesion of the composition is weak and it may be difficult to incorporate an adequate amount of the active ingredient. On the other hand, if the amount is greater than 120 μm, it is difficult to form a layer of the composition and to dry it.

The present invention is further illustrated by the following non-limiting Examples, of which Examples 1 to 3 illustrate the preparation of the adhesive copolymers and Examples 4, 5, 6 and Comparative Examples 1 to 10 illustrate the preparation and use of patches of the present invention.

### EXAMPLES 1-3

200 g of ethyl acetate (solvent), 0.05 g of azobisisobutyronitrile (initiator) and the monomers shown in the following Table 1 were charged into a reaction vessel, which was then sparged with nitrogen. Polymerisation was then carried out for 15 hours at 20° C. The resulting copolymer solution was coated with a knife coater onto a polyethylene terephthalate film to a dried thickness of 100 μm, and then dried at a temperature of 90° C. for 15 minute to produce adhesive sheets.

The resulting sheets were attached to the keratin layer of the shaven skin on the abdomen of Wistar rats. They were left there for 24 hours, after which they were removed, and the adhesiveness and state of the adhesive layer were observed with the naked eye. The results are shown in Table 1.

TABLE 1

| | | Amount | | | |
|---|---|---|---|---|---|
| | | of | | | |
| | Monomer | monomer | | Adhesion | Cohesion |
| Example 1 | methoxyethyl acrylate | 43 | g | no | no residue |
| | lauryl acrylate | 38 | g | detachment | left on |
| | N-vinylpyrrolidone | 6 | g | within 24 | body |
| | acrylic acid | 3 | g | hours | |
| | hydroxyethyl acetate | 10 | g | | |
| Example 2 | methoxyethyl acrylate | 48 | g | no | no residue |
| | lauryl acrylate | 34 | g | detachment | left on |
| | N-vinylpyrrolidone | 15 | g | within 24 | body |
| | acrylic acid | 3 | g | hours | |
| Example 3 | methoxyethyl acrylate | 50 | g | no | no residue |
| | lauryl acrylate | 36 | g | detachment | left on |
| | N-vinylpyrrolidone | 5 | g | within 24 | body |
| | hydroxyethyl acetate | 10 | g | hours | |

### EXAMPLES 4 & 5 & COMPARATIVE EXAMPLES 1-10

Preparation of Adhesive Patch

Into the adhesive solution (copolymer solution) of Example 1 and into the adhesive solutions used in the Comparative Examples, the drug specified below, and, where used, isopropyl myristate and/or a crosslinker, were added and dissolved. The resulting compositions containing the dissolved drug were coated onto a poly(ethylene terephthalate) film to give a thickness when dry of 100 μm, and dried at a temperature of 90° C. for 15 minutes, to produce drug-containing adhesive sheets.

The adhesives used for the Comparative Examples were obtained from National Starch, and are sold under the designations DT2287, DT2516, DT2051, DT2052, and DT2074.

The compositions of the adhesive mixtures are shown in Tables 2 and 3.

TABLE 2

| | | Example No. | | | | | |
|---|---|---|---|---|---|---|---|
| Ingredient | | 4 | Comp 1 | Comp 2 | Comp 3 | Comp 4 | Comp 5 |
| Example | Solid, | 250 | — | — | — | — | — |
| No. 1 | mg | | | | | | |
| DT2287 | Solid, | — | 250 | — | — | — | — |
| | mg | | | | | | |
| DT2516 | Solid, | — | — | 250 | — | — | — |
| | mg | | | | | | |
| DT2051 | Solid, | — | — | — | 250 | — | — |
| | mg | | | | | | |
| DT2052 | Solid, | — | — | — | — | 250 | — |
| | mg | | | | | | |
| DT2074 | Solid, | — | — | — | — | — | 250 |
| | mg | | | | | | |
| IPM | mg | 100 | — | — | — | 100 | 100 |
| HAD | mg | 7.0 | 5.0 | 7.5 | 7.0 | 6.0 | 5.5 |
| Cross- | mg | 250 | — | — | — | — | — |
| linker* | | | | | | | |

*0.05% of aluminium acetylacetate/tetrahydrofuran solution.

TABLE 3

| | | Example No. | | | | | |
|---|---|---|---|---|---|---|---|
| Ingredient | | 5 | Comp 6 | Comp 7 | Comp 8 | Comp 9 | Comp 10 |
| Example No. 1 | Solid, mg | 250 | — | — | — | — | — |
| DT2287 | Solid, mg | — | 250 | — | — | — | — |
| DT2516 | Solid, mg | — | — | 250 | — | — | — |
| DT2051 | Solid, mg | — | — | — | 250 | — | — |
| DT2052 | Solid, mg | — | — | — | — | 250 | — |
| DT2074 | Solid, mg | — | — | — | — | — | 250 |
| IPM | mg | 100 | — | 100 | — | 100 | 100 |
| 7β-OH EPIA | mg | 8.0 | 6.5 | 12.0 | 10.5 | 7.0 | 9.0 |
| Crosslinker* | mg | 0.002 | — | — | — | — | — |
| PEG 400 | mg | 30 | — | — | — | — | — |

*0.05% of aluminium acetylacetate/tetrahydrofuran solution.

As shown in Table 2 and Table 3, the various adhesives were loaded with the saturation concentration of HAD or 7μ-OH EPIA. Where possible, isopropyl myristate (IPM) was used as an enhancer, since it is an excellent pharmaceutical excipient for the transdermal delivery of a drug because of its marked enhancing effect and low skin irritation. However, most of the conventional adhesives could not load enough IPM because it resulted in a decrease in cohesion.

### EXAMPLE 6

1. Saturation Concentration of HAD in Adhesives

The adhesive patches described in Example 4 and Comparative Examples 1-5 were mixed with progressively increasing concentrations of HAD and were stored (adhesive side up) at room temperature for 8 weeks. At the end of this time, the patches were examined to determine whether HAD had crystallised on the surfaces of the patches. The saturation concentrations of HAD in the adhesives are shown in Table 4.

TABLE 4

| Adhesives | Saturation of HAD (% of patch weight) |
|---|---|
| Example 4 | 2.0 |
| Comp 1 | 2.0 |
| Comp 2 | 3.0 |
| Comp 3 | 2.0 |
| Comp 4 | 1.5 |
| Comp 5 | 2.0 |

2. Saturation Concentration of 7β-OH EPIA in Adhesives

The adhesive patches described in Example 5 and Comparative Examples 6-10 were mixed with progressively increasing concentrations of 7β-OH EPIA and were stored (adhesive side up) at room temperature for 8 weeks. At the end of this time, the patches were examined to determine whether 7β-OH EPIA had crystallised on the surfaces of the patches. The saturation concentrations of 7β-OH EPIA in the adhesives are shown in Table 5.

TABLE 5

| Adhesives | Saturation of 7β-OH EPIA (% of patch weight) |
|---|---|
| Example 5 | 2.0 |
| Comp 6 | 2.5 |
| Comp 7 | 3.3 |
| Comp 8 | 4.0 |
| Comp 9 | 2.0 |
| Comp 10 | 2.5 |

3. Permeation Behaviour

The materials used were as follows:

| | |
|---|---|
| Animals: | Wistar rats (male, body weight 240˜250 g) |
| Test patches: | Examples 4 and 5 and Comparative Examples 1-10 |
| | Commercial estradiol patch as permeation marker |
| | (Estrana, Hisamitsu Pharmaceutical Co.) |
| Patch size: | 8 mm diameter for HAD and 10 mm diameter for |
| | 7β-OH EPIA |
| Receptor solution: | PEG400:water (1:3) |
| HPLC conditions | |
| Column | ODS 5 um, 4.6 × 150 mm |
| Mobile | water:acetonitrile (55:45) |
| Flow rate | 1 ml/min |
| UV detector | 240 nm for HAD, |
| | 300 nm for 7β-OH EPIA |
| Column temperature | 40° C. |

The excised abdominal rat skin was mounted on a Franz type cell (receiver volume: 3ml) with a water jacket connected to a water bath at 37° C. The patch was adhered to the stratum comeum side of the skin. The receiver compartment was filled with a mixture of PEG400 and water (1:3 by volume) and stirred with a star head magnetic bead driven by a constant speed motor. At predetermined times, 100 μl of sample was withdrawn from the receiver compartment, and the same volume of receptor solution was added to keep the volume constant. The drug concentration was analysed by HPLC.

For all adhesives, the amount of the drug which had permeated increased steadily with time. The results after 24 hours are shown in Table 6 and Table 7.

TABLE 6

| Adhesive | Permeated amount μg/cm² |
|---|---|
| Example 4 | 30 |
| Comp 1 | 28 |
| Comp 2 | 20 |
| Comp 3 | 30 |
| Comp 4 | 50 |
| Comp 5 | 60 |
| Estradiol | 15 |

TABLE 7

| Adhesive | Permeated amount μg/cm² |
|---|---|
| Example 5 | 162 |
| Comp 6 | 150 |
| Comp 7 | 110 |
| Comp 8 | 52 |
| Comp 9 | 74 |
| Comp 10 | 73 |
| Estradiol | 13 |

All patches showed that a much higher amount of HAD and 7β-OH EPIA had permeated than that of the permeation marker, estradiol. This means that HAD 7β-OH EPIA have a potential skin permeability.

It was surprisingly found that the skin permeation of HAD was best with adhesives having the functional group —COOH or a combination of the functional groups —COOH/—OH, which make the adhesive highly hydrophilic. This allows HAD to be released into the skin. Adhesives having just the —OH group, such as DT2516, did not allow good permeation, whereas adhesives with a combination of groups —COOH/—OH, such as Example 1 and DT2074, allowed good permeation. Allowing for experimental variations due to variations in individual rats, the adhesive of Example 1 is as good as, if not better than, the known adhesives DT2052 and DT2074 for HAD permeation.

4. Permeation in Human Skin

The protocol in 3 above was repeated with HAD on human skin using the composition of Example 4 (Table 2) together with 5% w/w of lactic acid as enhancer. A penetration flux of 1.1 μg/cm²/hr in human skin was achieved.

1. The use of compounds of formula (I): 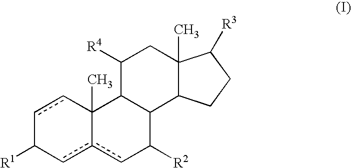
(in which R¹, R², R³ and R⁴ are the same as or different from each other and each represents an oxo group, a hydroxy group, a mercapto group, a hydrogen atom, a halogen atom, an alkoxy group or an aryloxy group; and the dotted line indicates that there may be a single or double bond between one or two of the respective non-adjacent pairs of carbon atoms) and esters thereof for the manufacture of a medicament for transdermal administration.

2. The use according to claim 1, in which said compound is a compound of formula (II): 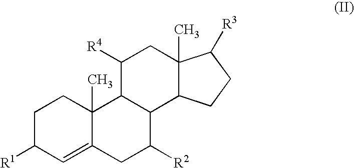
(in which R¹, R², R³ and R⁴ are as defined in claim 1) or an ester therof.

3. The use according to claim 1, in which said compound is a compound of formula (III): 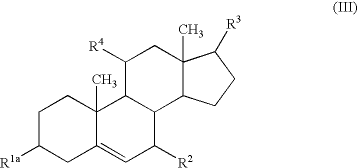
(in which R^{1a} represents an oxo group, a hydroxy group, a mercapto group or a halogen atom; and R², R³ and R⁴ are as defined in claim 1) or an ester thereof.

4. The use according to claim 1, in which said compound is a compound of formula (IV): 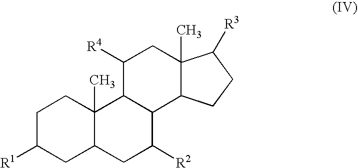
(in which R¹, R², R³ and R⁴ are as defined in claim 1) or an ester thereof.

5. The use according to claim 1, in which said compound is 11β-hydroxy-4-androstene-3,17-dione.

6. The use according to claim 1, in which said compound is 7α-hydroxy-dehydroepiandrosterone.

7. The use according to claim 1, in which said compound is 7β-hydroxy-epiandrosterone.

8. The use of compounds of formula (I): 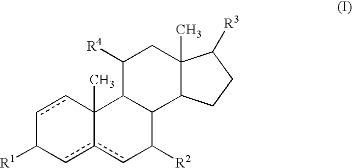
(in which R¹; R², R³ and R⁴ are the same as or different from each other and each represents an oxo group, a hydroxy group, a mercapto group, a hydrogen atom, a halogen atom, an alkoxy group or an aryloxy group; and the dotted line indicates that there may be a single or double bond between one or two of the respective non-adjacent pairs of carbon atoms) and esters thereof for the manufacture of a medicament for the treatment or prevention by transdermal application of osteoporosis, osteogenesis, neuronal cell death, or ischaemia-induced damage to peripheral organs.

9. The use according to claim 8, in which said compound is a compound of formula (II): 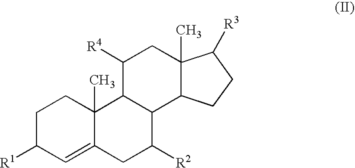
(in which R¹, R², R³ and R⁴ are as defined in claim 8) or an ester thereof.

10. The use according to claim 8, in which said compound is a compound of formula (III): 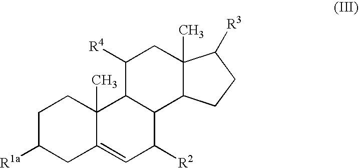
(in which R^{1a} represents an oxo group, a hydroxy group, a mercapto group or a halogen atom; and R², R³ and R⁴ are as defined in claim 8) or an ester thereof.

11. The use according to claim 8, in which said compound is a compound of formula (IV): 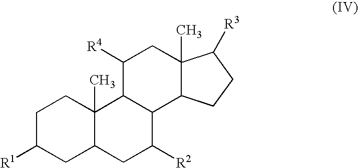
(in which R¹, R², R³ and R⁴ are as defined in claim 8) or an ester thereof.

12. The use according to claim 8, in which said compound is 11β-hydroxy-4-androstene-3,17-dione.

13. The use according to claim 8, in which said compound is 7α-hydroxy dehydroepiandrosterone.

14. The use according to claim 8, in which said compound is 7α-hydroxy-dehydroepiandrosterone.

15. An adhesive patch for the transdermal administration of a compound of formula (I): 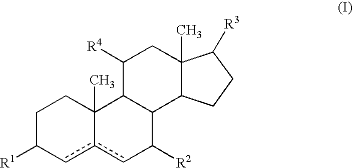
(in which R¹; R², R³ and R⁴ are the same as or different from each other and each represents an oxo group, a hydroxy group, a mercapto group, a hydrogen atom, a halogen atom, an alkoxy group or an aryloxy group; and the dotted line indicates that there may be a single or double bond between one of the respective pairs of carbon atoms) or an ester thereof, said patch comprising a substrate and a layer of adhesive on the substrate, said adhesive having dispersed therein at least one compound of formula (I) or an ester thereof.

16. A patch according to claim 15, in which said compound is a compound of formula (II): 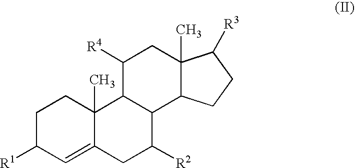
(in which R¹, R², R³ and R⁴ are as defined in claim 15) or an ester thereof.

17. A patch according to claim 15, in which said compound is a compound of formula (III): 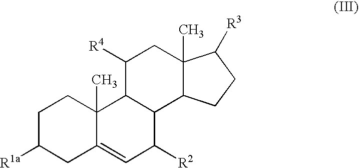
(in which R^{1a} represents an oxo group, a hydroxy group, a mercapto group or a halogen atom; and R², R³ and R⁴ are as defined in claim 15) or an ester thereof.

18. A patch according to claim 15, in which said compound is a compound of formula (IV): 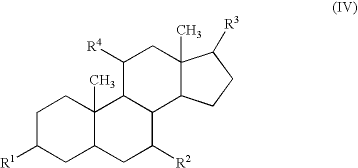
(in which R¹, R², R³ and R⁴ are as defined in claim 15) or an ester thereof.

19. A patch according to claim 15, in which said compound is 11β-hydroxy-4-androstene-3,17-dione.

20. A patch according to claim 15, in which said compound is 7α-hydroxy-dehydroepiandrosterone.

21. A patch according to claim 15, in which said compound is 7β-hydroxy-epiandrosterone

22. A patch according to claim 15, in which said adhesive comprises a copolymer of from 40 to 60% by weight of methoxyethyl acrylate, from 30 to 40% by weight of lauryl acrylate or methacrylate and from 10 to 25% by weight of a polar monomer.

23. A patch according to claim 22, in which the amount of units derived from methoxyethyl acrylate in the copolymer is from 45 to 55% by weight.

24. A patch according to claim 22, in which the polar monomer is acrylic acid, methacrylic acid, acrylamide, methacrylamide, N-vinyl-2-pyrrolidone, N,N-dimethylacrylamide, 2-hydroxyethyl acrylate or vinyl acetate.

25. A patch according to claim 24, in which the polar monomer is acrylic acid, N-vinyl-2-pyrrolidone or 2-hydroxyethyl acrylate.

26. A patch according to claim 25, in which the polar monomer is N-vinyl-2-pyrrolidone.

27. A patch according to claim 15, in which the adhesive additionally contains an absorption promoter.

28. A patch according to claim 27, in which said absorption promoter is isopropyl myristate.

29. A patch according to claim 27, in which said absorption promoter is present in an amount from 3 to 40 parts by weight per 100 parts by weight of the copolymer.

30. A patch according to claim 15, in which the adhesive layer has a thickness of from 30 to 120 μm.
